(19) United States
(12) Patent Application Publication
(10) Pub. No.: US 2009/0131854 A1
(43) Pub. Date: May 21, 2009

(54) Methods and Devices for Thermally Degrading Bacteria and Biofilm

(75) Inventors: Paul DiCarlo, Middleboro, MA (US); Stephanie Dubay, Dunstable, MA (US)

Correspondence Address: FINNEGAN, HENDERSON, FARABOW, GARRETT & DUNNER;LLP, 901 NEW YORK AVENUE, NW, WASHINGTON, DC, 20001-4413, US

(73) Assignee: Boston Scientific Scimed, Inc.

(21) Appl. No.: 12/271,620

(22) Filed: Nov. 14, 2008

Related U.S. Application Data
(60) Provisional application No. 60/988,163, filed on Nov. 15, 2007.

(51) Int. Cl. A61M 25/00

(52) U.S. Cl. 604/21

(57) ABSTRACT

Described herein are various implantable devices that include a heat generating element for degrading bacterial. In particular, the device can include a catheter having a heat generating element proximate to a distal end of the catheter for heating an outer surface of the catheter.

This application claims priority to U.S. Provisional Application No. 60/988,163, entitled “Methods and Devices for Thermally Degrading Bacteria and Biofilm,” filed Nov. 15, 2007, the contents of which is incorporated herein by reference.

### BACKGROUND

Catheters find a wide range of uses in modern medicine and allow medical personnel to deliver medication, drain fluids, monitor patient physiology, and access internal anatomy for the delivery of therapeutic and diagnostic devices. However, invasive medical devices, including catheters, can put patients at risk for bloodstream infection. In particular, infections, such as bacteremia or fungemia, are often associated with central venous catheters (CVCs).

In an effort to combat bloodstream infection, conventional catheters incorporate features to impede the spread and growth of bacteria. For example, aseptic hub devices such as puncture membranes inhibit the introduction of microbes into the catheter lumen. Catheters can also include antimicrobial materials coated thereon or impregnated therein. In addition, for long-term indwelling catheters, the delivery of anticoagulant/antimicrobial compounds is often prescribed.

Such preventative methods can reduce the chance of bacterial colonization. However, once established, microorganisms can adhere to a catheter surface and maintain themselves by producing a microbial biofilm. The organisms embed themselves in the biofilm layer, and can become resistant to antimicrobial agents and antibiotics. The structure and composition of microbial biofilm can inhibit the activity of some antibiotics and can insulate microbes from antibacterial agents embedded in the catheter, making traditional treatments less effective. Thus, the biofilm can both encourage bacterial growth and limit the effectiveness of antimicrobial treatments.

The biofilm can also lead to the build up of other biologically active substances produced by the body or microbes and trapped in the biofilm. In particular, bacteria associated with biofilm can produce thrombosis-inducing proteins. Thus, controlling the growth of biofilm and bacteria can also help to reduce the occurrence of catheter occlusion.

Thus, while some conventional catheters include features to reduce the growth of biofilm, the need still exists for additional methods of protecting medical devices against the growth of bacteria, the formation of biofilm, and the occurrence of occlusions.

### SUMMARY

Described herein are systems and methods for protecting against bloodstream infection associated with implantable devices. Unlike conventional devices that rely upon antibacterial materials and/or antibiotic solutions, the devices described herein use heat to degrade bacteria and biofilm. For example, the implantable device can include a heating element (also referred to herein as a heat generating element) for delivering heat to an implanted surface of the device.

In one embodiment the implantable device is a catheter that includes an elongate body extending from a proximal end to a distal end and having an inner lumen and an outer surface. The inner lumen can extend between a proximal opening and a distal opening. The body can further include a heat generating element adapted to heat the outer surface of the catheter.

Generally, the heat generating element is adapted to heat the outer surface of the catheter to a temperature sufficient to at least damage the bacteria while having a minimal impact on surrounding tissue or blood (e.g., without causing blood clotting). In one aspect, the heating element can be configured to heat the outer surface of the catheter to a temperature in the range of about 50 and 70° C., and in another aspect to a temperature in the range of about 55 and 65° C.

The heat generating element can also be configured to heat the outer surface of the catheter without causing damage to the catheter walls. The elongate body can be formed from typical catheter materials. In one exemplary aspect, the catheter body is formed materials, such as, for example, silicones, polyurethanes, polyethylenes, polyamide-polyesters, fluoropolymers, and combinations thereof. Where the catheter adjacent to the heat generating element is formed from thermoplastic polyurethanes and/or other high-temperature-application polymers, the maximum temperature of the heating element can be less than about the melt temperature of the adjacent catheter body. For other thermoset polymers, homopolymer, copolymer, and/or miscible thermoplastic polymer blends, the maximum temperature can be less than about the melt temperature of the material.

The heat generating element can be defined by a variety of electrically conductive structures. In one embodiment, the heat generating element is positioned within a sidewall of the catheter. For example, the heat generating element can be a coil defined by one or more conductive filaments embedded in the catheter. The embedded coil can heat the outer surface of the catheter adjacent to the distal opening. In addition, or alternatively, the heat generating element can heat the inner surface of the catheter adjacent to the distal opening, and kill any biofilm ingrowth that may occlude the distal opening of the catheter.

In another aspect, the heat generating element is defined by a tubular body or cylindrical body positioned within the sidewall of the catheter. In yet another aspect, the heat generating element is defined by longitudinally extending bands configured to heat the outer surface of the catheter. In still another aspect, the heat generating element can be defined by a conductive polymer, ink, or metal deposited in, mated with, or embedded in the sidewall of the catheter.

In another aspect, the heat generating element is defined by at least a portion of a reinforcing braid. The distal heat generating element have a higher electrical resistance than a proximal portion of the braid. For example, the braid can comprise proximally positioned filaments having a larger diameter than distally positioned filaments. Similarly, proximally positioned filaments can be formed of materials having a lower electrical resistance than the distally positioned filaments. When an electrical current is delivered to the reinforcing braid, the distal portion of the braid can heat to a temperature sufficient to degrade bacteria.

In still another embodiment, the heating element can be positioned on the outer surface of the catheter body. For example, an electrically conductive material can be positioned on the outer surface of the catheter and include the various configurations of the embedded heat generating elements described above.

The heat generating element can be in electrical communication with an electrical control unit. For example, an electrical wire can extend from a proximal hub of a catheter along, or through, the catheter body to the heat generating element. The electrical control unit can vary electrical power delivered to the heating element to control the temperature of the heating element. In addition, the electrical control unit can communicate with sensors associated with the catheter to control power delivery based on sensed temperature.

In another aspect, the elongate body includes a receiver for receiving energy via radio frequency (RF) induction. Power can be delivered to the heat generating element without the need to for a transmission wire. For example, an electrical current can be generated in an RF induction receiver positioned in the catheter and then transmitted to the heat generating element. Alternatively, the heat generating element can act as the receiver.

In another embodiment, a vascular catheter device is disclosed. The catheter includes an elongate body shaped and sized for at least partial insertion through a vascular lumen. The elongate body can extend from a proximal end to a distal end and have a sidewall between an inner lumen and an outer surface. The catheter can further comprise a heat generating element, wherein the heat generating element is adapted to degrade bacteria by heating the outer surface of the catheter.

In yet another embodiment, a central venous catheter device is disclosed. An elongate central venous catheter body can extend from a proximal hub to a distal end and have a sidewall between an inner lumen and an outer surface. The inner lumen can extend between the proximal hub and an opening proximate to the distal end of the catheter body. The catheter body can further comprise a heat generating coil proximate to the distal end of the catheter body, wherein the coil extends around the inner lumen and is adapted to heat an outer surface of the central venous catheter to a temperature sufficient to at least partially degrade bacteria.

In still another embodiment, a method of degrading a biofilm or bacteria is provided. The method can include providing an elongate catheter extending between a proximal and distal end, the catheter including an inner lumen, an outer surface, and a sidewall therebetween. The catheter can include a heating element proximate to the distal end. The method can further comprise the steps of placing the catheter at a target anatomic location. The heating element can be actuated to degrade a biofilm located on the outer surface of the catheter without damaging adjacent tissue. The heating step can be performed once a biofilm starts to grow, an occlusion is formed, or as needed to prophylactically inhibit growth and propagation of a biofilm.

The step of heating can include delivering electrical energy to the coil or heating element. In one aspect, the electrical energy is delivered via a wire or conductive element extending from the catheter hub to the heating element. In another aspect, the power is delivered via RF induction.

The step of heating can include degrading a biofilm without significantly raising the temperature of adjacent blood or tissue. The catheter can be heated with a known, safe power lever and/or the catheter temperature can be controlled via an algorithm based on variables, such as, for example, temperature, power, and/or time. In addition, or alternatively, a temperature feedback and sensor system can be used. In addition, or alternatively, a cooling fluid can be delivered through the inner lumen during the step of heating.

The method can additionally comprise the step of using the heating element to determine the location of the catheter using an imaging technique. For example, the location of the heating element can be determined via x-ray, MRI, CT, PET, SPECT, thermal/infrared, and/or fluoroscopy.

### BRIEF DESCRIPTION OF THE DRAWINGS

The accompanying drawings, which are incorporated in and constitute a part of this specification, provide illustrative embodiments, and, together with the description, serve to explain the principles of the disclosed devices and methods.

FIG. 1 is a perspective view of an embodiment of a system for heating a catheter to degrade bacteria;

FIG. 2 is a partial perspective view of an embodiment of a catheter described herein;

FIG. 3A is a partial side view of another embodiment of a catheter described herein;

FIG. 3B is a partial side view of another embodiment of the catheter of FIG. 3A;

FIG. 4 is a partial perspective view of another embodiment of a catheter described herein;

FIG. 5 is a cross-sectional view of yet another embodiment of a catheter described herein; and

FIG. 6 is a cross-sectional view of still another embodiment of a catheter described herein.

### DETAILED DESCRIPTION

Disclosed herein are methods and devices for degrading bacteria, fungus, and/or a biofilm positioned on a surface of a catheter. As used herein, the term “degrading” bacteria or biofilm refers to the inactivation or destruction of bacteria. In one aspect, a heat generating element heats the surface of the catheter to degrade bacteria while minimizing blood clotting and/or tissue damage. For example, the catheter can include an elongate body having an inner lumen configured for the delivery and/or removal of a fluid and/or configured for providing access to an anatomic site. When a biofilm or microbial deposit forms on the inner and/or outer surface of the catheter, energy is delivered to the heat generating element to degrade the biofilm or microbial deposit.

A variety of conventional procedures exist for limiting bacterial growth along exposed portions of a catheter. However, even with such procedures, a biofilm can sometimes form on an implanted catheter. In particular, the outer surface of an implanted catheter is particularly difficult to clean because antimicrobial fluid delivered through the catheter lumen does not reach the outer surface of the catheter. Similarly, the effectiveness of antimicrobial materials embedded in the catheter have a limited duration of effectiveness because once the antimicrobial materials are released they cannot be reloaded.

Unlike conventional catheters, the methods and devices described herein employ heating to degrade and/or destroy biofilm positioned on the outer surface of a catheter, particularly biofilm positioned on a distal, implanted portion of the catheter. Heating an implanted device could theoretically result in unwanted blood clotting and/or tissue damage. However, the catheter of the present invention uses low power/temperature heating to degrade a biofilm. In fact, the biofilm can act as a heat insulator such that the presence of biofilm helps to minimize the chance of blood clotting and/or tissue damage.

A variety of catheters can be used with the heating element described herein. However, in one embodiment the heating element is positioned in a catheter designed for long-term implantation. For example, the heating element can be employed with a range of catheters, such as (but not limited to), for example, hemodialysis, peritoneal, infusion, PICC, CVC, and port type catheters and for catheter applications including surgical, diagnostic and disease treatments. It is contemplated that the catheter can be used for administration of fluids (e.g., medication), for sampling or withdrawing bodily fluids (e.g., blood, urine, etc.), and/or for testing a condition of the body (e.g., blood pressure).

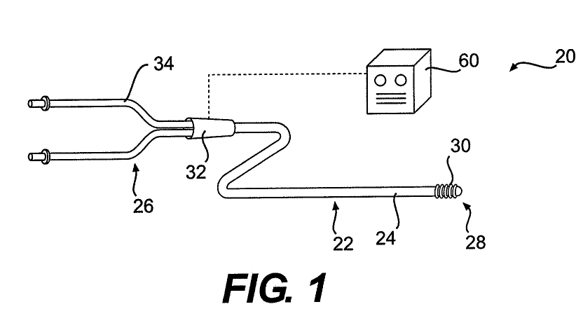

FIG. 1 illustrates one exemplary embodiment of a system 20 for delivering thermal energy to the outer surface of a catheter 22. Catheter 22 includes an elongate body 24 extending between a proximal end 26 and a distal end 28. A heating element 30, positioned proximate to the distal end of catheter 22, delivers thermal energy for degrading a biofilm, bacteria, and/or fungus.

The proximal end of catheter 22 can include at least one aperture for ingress into an inner lumen. A catheter hub 32 provides a housing that defines a pathway(s) into elongate body 24 and can provide a surface for mating with patient. In addition, the catheter hub can mate with extensions 34 that define pathways into hub 32 for receipt of medical devices and/or therapeutic agents. While the illustrated hub is not implantable, in another aspect, all or a portion of hub 32 could be configured for implantation. One skilled in the art will appreciate that the variety of conventional catheter hubs can be used with system 20.

The elongate body 24 of catheter 22 can house at least one inner lumen for transporting fluid and/or delivering a medical device. In one aspect, body 24 can include two, or more than two inner lumens. In addition, one or more of the inner lumens can extend for less than the full length of the elongate body. For example, two inner lumens can converge or an inner lumen can terminate prior to the distal end of elongate body 24.

Body 24 can be formed from a variety of materials, including the variety of biocompatible, flexible materials used with conventional catheters. One skilled in the art will appreciate that the choice of materials will depend on the intended use of catheter 22, and can include materials, such as (but not limited to), for example, silicone, polyurethane, polyethylene, polyamide-polyester, fluoropolymer, hydrogels, and combinations thereof. In addition, different sections of the catheter body can include different materials, such that the properties of the catheter changes along its length or width. In one such configuration, the distal portion of the catheter can include less rigid materials compared with a proximal portion of the catheter.

In addition, the catheter body walls can include more than a single layer of material. For example, the sidewalls of the catheter body can include one, two, three, or more than three layers of material which are mated to one another. In one aspect, the catheter body walls further include reinforcing materials to help strengthen the catheter and/or to vary a catheter body property (e.g., stiffness). In one such aspect, reinforcing filaments, patterned for example in a braided configuration, can extend along a portion of the catheter body.

The size (length and diameter) and shape of elongate body 24 can be chosen depending on the desired insertion site, access site, and/or distal tip implantation site of the catheter. In one embodiment, catheter 22 and elongate body 24 are sized and shaped for venous access. For example, catheter 22 can be configured for insertion through an upper extremity, jugular vein, or subclavian vein. Where catheter 22 is intended for insertion through a large vein of the peripheral vascular system, the outer diameter of elongate body 24 can have a diameter in the range of about 2 French and 22 French, and in another aspect in the range of about 2 French and 10 French. Additionally, the catheter can have a varying diameter defined by, for example, a taper along at least a portion of the catheter.

As illustrated in FIG. 1, system 20 can also include a control unit 60 that can permit heating of catheter 22. In one aspect, control unit 60 is distinct from hub 32 and body 24, alternatively, the control unit can be built into a portion of catheter 22. A user interface, defined by a portion of the control unit, can allow a clinician to direct the delivery of energy, such as, for example, electrical current, to a heat generating element. The control unit is described in more detail below.

FIG. 2 illustrates a distal portion 40 of catheter 22 including at least one opening 38 to an inner lumen 42 for the ingress and/or egress of fluid. In one aspect the distal opening 38 can be positioned at the distal-most end of the catheter. Alternatively, or additionally, catheter 22 can include a distal opening spaced from the distal-most end of the catheter. For example, catheter 22 can include an opening (not illustrated) in the sidewall of the catheter body 24.

The size and shape of the distal portion 40 of the catheter body can vary depending on the intended use of the system. For example, the distal portion of the catheter body can be sized and shaped for placement in or adjacent to an anatomical structure. In one aspect, the distal portion 40 of the catheter body is sized for placement within vascular structure, such as, for example the superior vena cava.

The distal portion 40 of catheter 22 further includes heating element 30. After implanting catheter 22, a biofilm can grow on the outer surface of the catheter. In order to degrade the biofilm, heating element 30 can raise the temperature of the biofilm. In particular, the heating element can raise the temperature of the biofilm enough to degrade pathogens, while preventing damage to the catheter and surrounding anatomy. Table 1 lists biofilm bacteria commonly attributed to catheter-related blood stream infections.

TABLE 1

| Pathogen | Species |
|---|---|
| coagulase-negative staphyococci | Staphylococcus aureas |
| | Pseudomonas aeruginosa |
| | Enterococcus faecalis |
| | Candida Species |
| | Staphylococcus epidermidis |
| Candida Albicans | |
| Aerobic gram-negative bacilli | E. Coli |
| | Klebsiella |
| | Enterobacter |
| | S. marcescens |
| | acinetobacter |
| gra-positive cocci | S. aureas |

Heating, for at least some of the above referenced bacteria, results in thermal inactivation of the bacteria and a reduction in bacteria concentration. In one aspect, the heating element heats the outer surface of the catheter and/or biofilm to a temperature in the range of about 50 and 75° C., and in another aspect, in the range of about 55 and 70° C., and in yet another aspect, in the range of about 55 and 65° C. The chosen temperature range can be selected, for example, depending on the length of time which the catheter is heated, the anatomic site of the catheter, the materials of the catheter, the chosen heating element or heating element configuration, and/or the intended use of the catheter.

The structure of biofilm can assist with degrading the bacterial contained therein. When the heating element raises the temperature of the outer surface of the catheter (which in one aspect, can be the outer surface of the heating element), the temperature of the biofilm rises. The amount of heating can be controlled such that a temperature gradient across the biofilm and/or catheter wall heats the biofilm within the desired temperature range without overheating adjacent blood or tissue (e.g., causing blood clotting or lesion formation). In addition, depending on the placement of the catheter, the flow of blood passing over the catheter carries away the heat energy at the catheter and/or biofilm blood interface. Because the blood is continuously moving, the blood does not have a chance to heat to a temperature sufficient to cause coagulation.

The heating element can be defined by a metal or polymeric conductive body that extend over, extends through, and/or defines at least a portion of the catheter sidewall. Heating of the biomaterial to a desired temperature depends on the resistive properties of the material, the thermal diffusivity constant of the material, the shape of the heating element, the volume of the heating element, the position of the heating element (and intervening structure) relative to the outer surface of the catheter, and the power delivered to the catheter. Thus, the various aspects of the heating element described below can be varied to achieve an effective heating element.

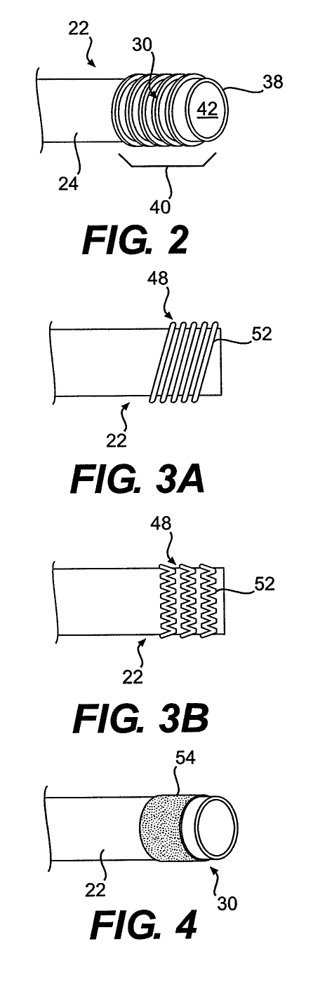

In one embodiment, the heating element has a coil configuration defined by strands or filament that wind around a portion of the catheter body. FIGS. 3A and 3B illustrate exemplary embodiments of coil 48 defined by filaments 52 wound around the outer surface of the distal portion of the catheter body 22. In one aspect, as shown in FIG. 3A, a filament winds around the outer surface of catheter 22 without crossing other filaments. The pitch of the winding and the spacing between the filaments can be varied depending on desired temperature, location of the filaments, power delivered to the filaments, and the intended use of the system 20. For example, the filaments can be positioned immediately adjacent to one another or spaced from one another. In another aspect, filament 52 zig-zags as it passes around the outer surface of the catheter body. In addition, multiple filaments can define coil 48, including multiple coils that cross one another or have different patterns or materials properties from one another. In yet another aspect, one or more of the filaments can be oriented longitudinally. The filament can be formed of the variety of electrically conductive and/or resistive materials, including metals, polymers, and ceramics.

In another aspect, instead of a filament or filaments, heating element 30 is defined by a band or tubular body. FIG. 4 illustrates a tubular body 54 extending around the outer surface of catheter body 22. In another aspect, tubular body 54 could include apertures and/or multiple tubular bodies could be used. In addition, or alternatively, the tubular body could be patterned (for example, by etching or other such patterning processes).

Heating element 30, regardless of its configuration, can mate with the catheter in a variety of ways. In one aspect, the heating element adheres or mechanically engages the outer surface of the catheter body. In another aspect, the heating element can be partially positioned within the outer wall. For example, the heating element could be seated within a recess in the outer surface of the catheter or partially embedded within the catheter wall.

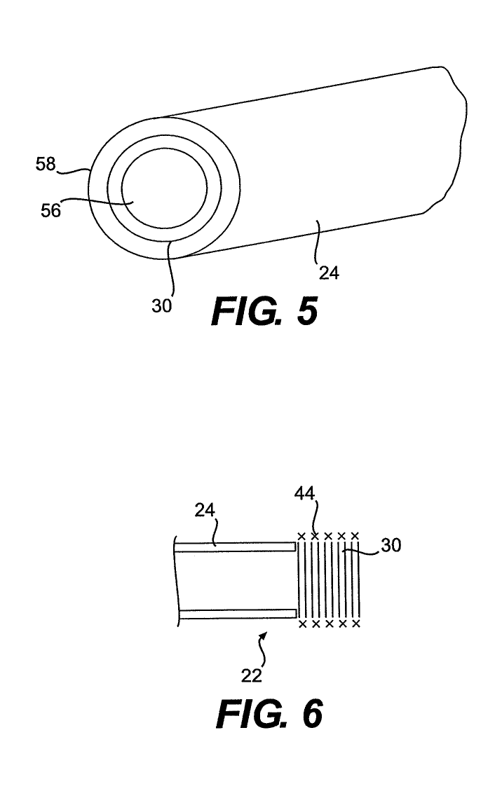

Fully embedding the heating element within the sidewalls of catheter 22 provides an alternative configuration of system 20. FIG. 5 illustrates a cross-sectional view of the distal portion of catheter body 22. Heating element 30 is positioned between an inner and outer surfaces 56, 58 of the catheter. In particular, heating element 30 is embedded within the catheter sidewall. Where the catheter body has multiple inner lumens and internal walls between the inner lumens, the heating element can be embedded in the outermost sidewall of the catheter.

In one aspect, the catheter sidewall is formed from multiple layers and the heating element is embedded between layers. Where the heating element is defined by filaments, the filaments can be formed on the outer surface of an inner layer, and an outer layer can then be formed over the coil. Alternatively, the coil can be pressed into the sidewall of the catheter or co-extruded with the sidewalls. Similarly, where the coil is defined by a tubular body, the catheter sidewalls can be formed around the tubular body or the tubular body can be inserted into a pre-formed catheter. In yet another embodiment, an electrically conductive metal can be deposited in or on the catheter sidewall. One skilled in the art will appreciate that the heating element can be incorporated during a variety of conventional catheter forming techniques. In another embodiment, the heating element can be formed by a conductive ink coating and/or conductive nanoparticle coating.

In another embodiment, the heating element is formed by an electrically conductive polymer. For example, the electrically conductive polymer can be formed as an additional layer in or on the sidewall of the catheter. Applying electrical energy to the polymer heats the outer surface of the catheter. Alternatively, or additionally, an electrically conductive polymer can replace a layer of a catheter sidewall or can define the catheter sidewall.

When heated, heating element 30 heats the outer surface of the catheter to remove and/or degrade biofilm. The coil can also heat an inner surface of the catheter to degrade bacteria or fungus positioned thereon. However, in one aspect, the heating element can heat the outer surface of the catheter more than the inner surface of the catheter. For example, the heating element can be positioned closer to the outer surface of the catheter than the inner surface. Additionally, or alternatively, the catheter sidewall can be designed to transmit heat faster from the coil toward the outer surface compared with heat transfer from the coil toward the inner surface of the catheter. For example, the material forming the outermost surface of the catheter sidewall can have a higher thermal conductivity than the material forming the sidewall between the coil and the inner surface of the catheter.

Regardless of the location of the coil within or on the catheter body, heating element 30 can be positioned adjacent to the distal end of the catheter and/or adjacent to a distally positioned catheter opening. In one aspect, the heating element extends proximally from the distal-most end of the coil as shown in FIGS. 2 through 5.

In another embodiment, at least a portion of the heating element extends distally from the distal end of the catheter body. FIG. 6 illustrates catheter 22 with heating element 30 defining the distal portion of the catheter. Having the coil abutting catheter body 24 can reduce the amount of heat transferred to the catheter body when the coil is heated. The distally extending heating element can have a similar structure to the exemplary heating elements discussed above, including a coil or tubular body configuration. In one exemplary embodiment, the distally extending heating element is formed by a coil. To provide structure to the coil, the inner surface of the coil body can include a sheath or reinforcing element. For example, between the inner lumen defined by the distally extending coil and the filaments of the coil, system 20 can include a polymer sheet that mates with the filaments of the coil. Alternatively, or additionally, the filaments of the coil can be held together with an adhesive.

System 20 can further include a power source in communication with the heating element. In one aspect, as illustrated in FIG. 1, the power source is associated with a control unit 60 that is in electrical communication with heating element 30. In one aspect, the control unit is an on/off switch that when activated, causes the heating element to heat the surface of the catheter. In another aspect, the control unit can include a processor. Depending on the configuration of the heating element and the desired heating element temperature, the processor of control unit 60 can select an electrical current to the heating element. The processor can be programmed to cause delivery of a current of a chosen magnitude for a chosen time period or periods. For example, the processor can cycle the heating element on and off to periodically heat the catheter. The control unit can include a user interface and memory so that a user can program the control unit and/or can select between heating regimens stored in the memory.

In still another embodiment, the catheter can include a sensor to allow feedback. The sensor, for example, a temperature sensor, can provide the processor with temperature data and allow the processor to adjust the current delivered to the heating element. The temperature sensor (not illustrated) can be located at a variety of locations along, within, and/or on the catheter to allow the processor to determine a temperature profile. In addition, temperature data can be used by the processor to ensure that the temperature of blood or tissue surrounding the catheter does not exceed a maximum temperature and/or that the temperature does not rise to a level that could damage the catheter.

As described with respect to control unit 60, the power source can be positioned remotely from the catheter. The power source can be connected to the proximal portion of the catheter (e.g., catheter hub 32) via a transmission wire (not illustrated). Alternatively, the power source can be built into a portion of the catheter, such as, for example, the catheter hub. In one aspect, the power source is a rechargeable battery mated with a proximal portion of the catheter. The battery can be periodically recharged to provide power to the heating element.

Regardless of the location of the power source, energy can be transmitted between the proximal portion of the catheter and a distally positioned heating element in a variety of ways. In one aspect, a transmission wire can extend through the catheter to connect the power source to the heating element. The transmission wire can be housed within a catheter lumen, embedded within the sidewall of the catheter, and/or can extend along the surface of the catheter body. While the heating element is generally described in terms of resistive heating based on delivery of an electrical current, other heat sources can be used. For example, electromagnetic energy can be delivered to heat a portion of the catheter. In one such embodiment, energy can be delivered via a fiber optic cable.

As mentioned above, the catheter body can include a reinforcing braid extending through the catheter. Where the braid is formed of an electrically conductive material, the braid can transmit electrical energy between the proximal hub and the heating element. For example, U.S. Patent Application Publication No. 2005/0020965 to Rioux et al., which is incorporated by reference, describes a reinforcing braid that transmits energy through a medical device to an electrode.

In addition, the heat generating element can be defined by at least a portion of a reinforcing braid. The portion of the reinforcing braid acting as the heating element can have different properties from the other portions of the braid. In one aspect, a distal portion of the braid can be formed from materials having a higher electrical resistance, such that the distal portion of the braid heats more than a proximal portion of the braid when electrical current flows through the reinforcing element. Increased resistivity can be achieved, for example, by varying the size of the braid fibers and/or varying the materials that form the braid. In one aspect, the electrical resistance of the distal portion of the braid can be increased by the presence of fibers having a smaller cross-sectional area. In another aspect, the braid can have a higher braid density at the distal end of the catheter. One skilled in the art will appreciate that a variety of braid characteristics can be adjusted to achieve selective heating over a portion of the catheter. In addition, the braid need not be a “reinforcing” braid. The braid can be configured to generate heat without significantly adding to the torsional strength of the catheter.

The energy delivered to the heating element can have a monopolar or biopolar-type configuration. Thus, in one aspect, the catheter can include an energy delivery transmission wire and a return or ground wire. In addition, the current delivered to the heating element can be alternating or direct. As mentioned above, the power delivered to the heating element will depend on the heating element configuration (e.g., electrical resistance, size, location, etc.) and on the target temperature of the heating element. In one aspect, the catheter surface is heated to a temperature in the range of about 40 and 100 degrees Celsius, in another aspect, to a temperature in the range of about 50 and 70 degrees Celsius, and in yet another aspect, to a temperature in the range of about 55-65 degrees Celsius. Reducing bacteria concentration depends both on the temperature and the duration of the elevated temperature. An example of the amount of time required to kill common bacteria at a given temperature is described by R. H. Dunstan et al., Thermal Inactivation of Water-Borne Pathogenic Indicator Bacteria at Sub-Boiling Temperatures, Water Research, Volume 40, Issue 6, March 2006, pp. 1326-1332, the contents of which are incorporated herein by reference.

In another embodiment, the heating element can be heated wirelessly via electromagnetic induction. Wireless transmission of energy to the heating element can eliminate the space required for transmission wires extending through the catheter and allow a smaller diameter catheter. The power source can include a transmitter having a (RF) current passing through a coil of wire. The transmitter can magnetically couple to a receiver. For example, the receiver could be defined by a coil of electrically conductive wire. Running RF current through the transmitter generates an inductive current in the receiver that can power the heat generating element. For example, the receiver could receive power from the transmitter and direct the energy to the heating element.

In addition, the current generated in the receiver could power a sensor, such as, for example, a temperature sensor. Thus, when activated, the inductive current can power the heating element and a sensor, which allows feedback control of the heating element. In addition, or alternatively, the current generated by the receiver can supply power to a battery mated with the catheter. Thus, the inductive transfer of power and the heating of the heating element do not have to occur simultaneously.

In an alternative configuration, the heating element can act as the receiver. The coil can have a coil configuration that magnetically couples to the transmitter. When RF current is passed through the transmitter coil, a RF current can be generated in the heating element, causing the temperature of the heating element to rise.

To protect against accidental heating, the catheter can include an internal switch. When turned off, the switch can prevent accidental heating should a patient encounter an inductive field. The switch can be engaged prior to transmitting energy via RF induction. A variety of switches, such as mechanically or magnetically activated switches can prevent accidental heating.

While the heating element is generally described in terms of resistive heating based on delivery of an electrical current, other heat sources can be used. For example, electromagnetic energy can be delivered to heat a portion of the catheter. In one such embodiment, energy can be delivered via a fiber optic cable.

Further provided herein are methods of degrading bacteria or biofilm. The method can include the steps of providing an elongate catheter that includes a heating element proximate to the distal end and heating the heating element to degrade a biofilm positioned on an outer surface of the catheter without damaging adjacent tissue.

The step of heating can be achieved without damaging the catheter or causing significant blood clotting. In one aspect, the step of heating can include heating the outer surface of the catheter adjacent to the heating element to a temperature in the range of about 40 and 100° C., and in another aspect, in the range of about 50 and 70° C., and in yet another aspect, in the range of about 55 and 65° C.

To assist with preventing catheter damage, a cooling fluid can be delivered through the inner lumen during the step of heating. For example, saline solution, medication, antimicrobial solution, and/or other fluid can be delivered at the same time as the catheter is heated.

Prior to heating, the method can include the step of implanting the catheter. As part of inserting the catheter, a surgeon may wish to confirm the location of the distal end of the catheter. In one aspect, the heating element can act as a marker to allow visualization of the catheter. For example, the method can further comprise the step of using the heating element to determine the location of the catheter with an imaging technique. Exemplary imaging techniques include, for example, x-ray, MRI, CT, PET, SPECT, and/or fluoroscopy.

Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

1. A catheter device, comprising:
a catheter sized and shaped for vascular access and including an elongate body extending from a proximal end to a distal end and having an inner lumen and an outer surface, the inner lumen extending between a proximal opening and a distal opening, the body further including a sidewall and a heat generating element positioned therein, wherein the heat generating element is adapted to heat the outer surface of the catheter to a temperature sufficient to degrade bacteria and/or biofilm.

2. The device of claim 1, wherein the heat generating element is a coil contained within the sidewall.

3. The device of claim 2, wherein the coil heats the outer surface of the body adjacent to the distal opening.

4. The device of claim 2, wherein the elongate body extends to a distal tip and the coil is adapted to heat the distal tip.

5. The device of claim 1, wherein the heat generating element is defined by at least a portion of a reinforcing braid.

6. The device of claim 5, wherein the braid comprises proximally positioned filaments having a larger diameter than distally positioned filaments.

7. The device of claim 5, wherein the braid comprises proximally positioned filaments having a lower electrical resistance than the distally positioned filaments.

8. The device of claim 1, wherein the heat generating element is in electrical communication with an electrical control unit.

9. The device of claim 1, wherein an electrical wire extends along the body to the heat generating element.

10. The device of claim 9, wherein the electrical wire is form of a material having a lower electrical resistance than a material used to construct the heat generating element.

11. The device of claim 1, wherein the elongate body includes a receiver for receiving energy via RF induction.

12. The device of claim 11, wherein the receiver is in electrical communication with the heat generating element.

13. The device of claim 11, wherein the heat generating element acts as the receiver.

14. The device of claim 1, wherein the heat generating element is adapted to heat the outer surface of the body without causing blood clotting.

15. The device of claim 1, wherein the heat generating element is adapted to allow a user to determine the position of the distal end of the catheter using a radiological technique.

16. The device of claim 1, further comprising multiple inner lumens.

17. The device of claim 1, wherein a portion of the outer surface is heated to a temperature in the range of about 50 and 70° C.

18. The device of claim 1, wherein the elongate body is formed of a flexible material.

19. A catheter device, comprising:
an elongate body shaped and sized for at least partial insertion through a vascular lumen, the elongate body extending from a proximal end to a distal end and having a sidewall, the inner lumen extending between a proximal opening and a distal opening; and
a heat generating element, wherein the heat generating element is adapted to degrade bacteria by heating an outer surface of the catheter without ablating or damaging adjacent tissue.

20. The device of claim 19, wherein the elongate body is formed from a flexible material.

21. The device of claim 20, wherein the flexible material is selected from the group of thermoplastics, thermosets, engineering thermoplastics, and combinations thereof.

22. A central venous catheter device, comprising:
an elongate central venous catheter body extending from a proximal hub to a distal end and having a sidewall between an inner lumen and an outer surface, the inner lumen extending between the proximal hub and an opening proximate to the distal end of the catheter body, the catheter body further comprising a heat generating element proximate to the distal end of the catheter body, wherein the heat generating element is adapted to heat to a temperature sufficient to at least partially degrade bacteria.

23. The catheter of claim 22, wherein the heat generating element is a coil positioned within the sidewall.

24. The catheter of claim 22, wherein the heat generating element extends distally from the distal end of the catheter body.

25. A method of degrading a biofilm or bacteria, comprising:
providing an elongate catheter extending between a proximal and distal end, the catheter including an inner lumen, an outer surface, and a sidewall therebetween, the catheter further comprising a heating element proximate to the distal end; and
heating the heating element to degrade a biofilm positioned on the outer surface of the catheter without damaging adjacent tissue.

26. The method of claim 25, wherein the step of heating includes heating the outer surface of the catheter adjacent to the heating element to a temperature in the range of about 50 and 70° C.

27. The method of claim 25, wherein the step of heating includes degrading a biofilm without significantly raising the temperature of adjacent blood or tissue.

28. The method of claim 25, wherein the heating element is embedded in the sidewall.

29. The method of claim 25, wherein the step of heating includes delivering electrical energy to the heating element.

30. The method of claim 29, wherein the electrical energy is delivered via a wire extending to the heating element.

31. The method of claim 25, wherein the step of heating includes delivering power to the catheter via electromagnetic induction.

32. The method of claim 25, further comprising delivering a cooling fluid through the inner lumen during the step of heating.

33. The method of claim 32, wherein the cooling solution is saline solution.

34. The method of claim 25, further comprising the step of using the heating element to determine the location of the catheter using an imaging technique.

35. The method of claim 34, further comprising using x-ray, MRI, CT, PET, SPECT, and/or fluoroscopy to determine the location of the heating element.
